(19) United States
(12) Patent Application Publication
(10) Pub. No.: US 2007/0298462 A1
(43) Pub. Date: Dec. 27, 2007

(54) Process for Obtaining Antibodies

(75) Inventors: Mukesh Sehdev, Langley (GB); Mariangela Spitali, Maidenhead (GB)

Correspondence Address: SUGHRUE MION, PLLC, 2100 PENNSYLVANIA AVENUE, N.W., SUITE 800, WASHINGTON, DC, 20037, US

(73) Assignee: UCB Pharma S.A., Brussels (BE)

(21) Appl. No.: 11/791,108

(22) PCT Filed: Nov. 16, 2005
(86) PCT No.: PCT/GB05/04429

§ 371 Date: May 17, 2007

(30) Foreign Application Priority Data
Nov. 19, 2004 (GB) 0425537.8

(51) Int. Cl. C12P 21/04

(52) U.S. Cl. 435/69.6

(57) ABSTRACT

The present invention relates to the manufacture of recombinant antibodies of therapeutic quality. In particular, the invention relates to methods for increasig the yield of cuntional antibody from large scale fermentations whereby a cultured host cell sample is subjected to a freeze-thaw treatment step.

This invention relates to methods for increasing the yields in the production and isolation of functional recombinant antibodies, and in particular therapeutic antibodies. The methods are particularly suitable for the large-scale industrial manufacture of therapeutic antibodies.

Recombinant DNA techniques have rapidly developed and are particularly useful in the production of antibodies, in particular therapeutic antibodies. Systems for the expression of recombinant genes are well known to the person skilled in the field in question. These include expression in mammalian cells, insect cells, fungal cells, bacterial cells and transgenic animals and plants. The choice of expression system is dependent on the features of the encoded protein, for example post-translational modifications. Other considerations include the time and, in particular, the cost involved in the production of the desired quantity of material of the required quality. These latter considerations are particularly important in the production of therapeutic antibodies of the quality required for regulatory approval and in the quantities needed for treatment of large numbers of patients.

The most widely used system for the production of recombinant proteins is based on expression in Escherichia coli (E. coli). A specific problem encountered with the use of E. coli is the difficulty in producing material of the required quality in quantities need for therapy. In particular, the time and costs involved can be prohibitive. One specific problem of note is the loss incurred in the yield of antibodies during extraction of the antibodies from E. coli. A method that partially addresses this latter problem and that permits the production of antibodies acceptable for therapeutic use is described in U.S. Pat. No. 5,655,866. This method involves the use of heat treatment to facilitate the subsequent isolation of functional Fab′ fragments of antibodies from non-functional antibodies, the heat treatment being performed at any time during the fermentation or culture, or at any stage during extraction and purification of the antibodies. At elevated temperatures above room temperature, functional antibodies are remarkably stable, whilst many other proteins including host cell proteins and free light and heavy chain species and non-functional fragments of antibodies form precipitates and/or aggregates which are easily separated from functional antibody during primary purification procedures such as filtration or centrifugation or fluidised bed chromatography. Although proportionally, the purification costs are a fraction of the total cost of a therapeutic antibody product, the purification cost proportion will increase further as upstream production costs become cheaper. Thus, improvements in recovery and purification of antibodies will drive production costs down further irrespective of the means of production (Humphreys & Glover, Curr. Opin. Drug Discovery & Development, 2001, 4:172-185). Hence, there is a need for methods that introduce time and/or cost savings into therapeutic antibody production, and in particular in purification, for example by increasing yields.

Low yield per fermentation or culture is often a particular problem noted at the primary extraction stage; expression of antibody is high within the cells but a high percentage recovery at the primary extraction stage is remarkably difficult to achieve. U.S. Pat. No. 5,665,866 describes enhancement of initial purification yields by the inclusion of a heat treatment step which aids the purification process by removing non-functional antibody.

WO2005019466 (published after the priority date of this application) describes an increase in yield of recombinant proteins by the inclusion of an interruption step after fermentation but prior to downstream processing.

This invention described herein is based on the surprising and unexpected observation that freeze-thaw treatment in combination with heat treatment brings an increase in the yield of functional antibody at the primary extraction stage of up to 50%, i.e. the yield of functional antibody is increased above that of heat treatment alone. This enables hugely beneficial savings in time and cost of production of quantities of functional antibodies of therapeutic quality. It also lessens the impact of fermentation batch-to-batch variability, as fewer batches are needed to prepare the quantity required.

Accordingly, provided is a method for the manufacture of recombinant antibody molecules comprising culturing a host cell sample transformed with an expression vector encoding a recombinant antibody molecule and subjecting said sample to a freeze-thaw treatment step.

In a preferred example, the recombinant antibody molecule is at least part of an antibody light chain and at least part of an antibody heavy chain, such that at least some of the expressed light and heavy chain antibody molecules are able to combine to form functional antibody.

In a most preferred embodiment, the method further comprises subjecting the sample to an increase in temperature within the range of 30° C. to 70° C. for a period of up to 24 hours. Thus, the invention also provides a method of increasing the yield of functional antibody molecules isolated from a sample, said sample comprising soluble, functional antibody molecules, and non-functional antibody molecules, which method comprises subjecting the sample to an increase in temperature within the range of 30° C. to 70° C. for a period of up to 24 hours, said method characterised in that the sample is subjected to a freeze-thaw treatment step before being subject to the increase in temperature.

In particular, the method permits an increase in isolated functional antibody yields at a range of temperatures and treatment conditions, which can be varied as required, and understood by one skilled in the art, to take account of the particular characteristics of the functional antibody being produced and the expression system being used.

As used herein, ‘functional antibody’ includes antibody molecules that retain the ability to specifically recognise or bind to the antigen against which they were raised (cognate antigen). The production of a functional antibody is shown by the presence of a single band on non-reducing SDS-PAGE corresponding to the expected molecular weight of the antibody, or by direct binding assay using BIACore or other methods known to the person skilled in the art, for example but not limited to, ELISA. Non-functional antibodies include fragments which do not recognise their cognate antigen, and include incorrectly-folded or incorrectly-assembled antibodies, free heavy and light chains, and fragments thereof, including partially degraded fragments of antibodies which do not recognise or bind to their cognate antigen.

In the methods of the invention, a sample may be the product of a fermentation, for example but without limitation, a fermentation comprising bacteria, or yeast, a cell culture, for example but without limitation, a mammalian or insect cell culture. Most preferably, the sample is the product of a fermentation comprising E. coli expressing a recombinant antibody, wherein said antibodies may be functional and non-functional antibodies. If desired, the host cells may be subject to collection from the fermentation medium, e.g. host cells may be collected from the sample by centrifugation, filtration or by concentration. In particular, the methods of the invention are suitable for the large-scale industrial manufacture of antibodies of therapeutic quality.

Preferably, the host cells are collected from a fermentation or culture, e.g. by centrifugation, and placed at a temperature low enough to permit freezing of the cell sample. In one embodiment, the liquid cell sample is placed in a freezer at between −20° C. and −70° C. Preferably, the sample is frozen and stored at -20° C. Alternatively, the sample is frozen and stored at −70° C. Most preferably, freezing takes place slowly. “Freezing slowly” as used herein includes samples that are placed at a reduced temperature, for example placed in a freezer, and have not been snap frozen, for example on dry ice or in liquid nitrogen, before being placed in a freezer. In one embodiment, cell samples are snap frozen before being stored, e.g. in a freezer.

Alternatively, the host cells collected are suspended in a buffered solution using buffered salts such as, but not limited to, Tris, acetate or phosphate. The pH of the solution may, for example, be between pH 2 and pH 10 and will most preferably be between pH 6 and pH 8. A most preferred buffer is Tris buffer, pH 7.4 which may optionally further comprise EDTA, for example but without limitation, 100 mM Tris, pH 7.4 containing 10 mM EDTA before being subjected to a freeze-thaw treatment step.

In the methods of the invention, frozen samples may be kept frozen for any length of time suitable, for example 1 or 2 hours up to 1 or 2 weeks. In one embodiment, samples are kept frozen for between 2 to 4 hours before being allowed to thaw to room temperature. In another embodiment, samples are kept frozen for 2, 3 or 4 days before being allowed to thaw to room temperature. In yet another embodiment, samples are kept frozen overnight, for example for between 12 and 18 hours, before being allowed to thaw unassisted to room temperature. Alternatively, thawing may be assisted by, for example, using a water bath or warming oven.

Accordingly, provided is a method for the manufacture of recombinant antibody molecules comprising culturing a host cell sample transformed with an expression vector encoding a recombinant antibody molecule and subjecting said sample to a freeze-thaw step which consists of a slow freezing step and/or a slow thawing step. Thus, in one embodiment of the methods of the invention, provided is a method of increasing the yield of functional antibody molecules isolated from a sample comprising functional antibody molecules, and non-functional antibody molecules, which method comprises subjecting the sample to an increase in temperature within the range of 30° C. to 70° C. for a period of up to 24 hours, said method characterised in that the sample is subjected to a freeze-thaw step which consists of a slow freezing step and/or a slow thawing step before being subject to the increase in temperature.

Most preferably, heat treatment steps are performed within the range of 30° C. to 70° C. The temperature can be selected as desired and may depend on the stability of the antibody for purification. In another embodiment, the temperature is within the range 40° C. to 65° C., or preferably within the range 40° C. to 60° C., more preferably within the range 45° C. to 60° C., even more preferably within the range 50° C. to 60° C. and most preferably at 55° C. to 60° C. Thus, the minimum temperatures are 30° C., 35° C. or 40° C. and the maximum temperatures 60° C., 65° C. or 70° C. The length of heat treatment is preferably between 1 and 24 hours, more preferably between 4 and 18 hours, even more preferably between 6 and 16 hours and most preferably 10 and 14 hours, for example 12 hours. Thus, the minimum time for heat treatment is 1, 2 or 3 hours and the maximum is 20, 22 or 24 hours.

In a particular embodiment, the heat treatment is performed at 50° C. to 60° C. for 12 to 16 hours, and more preferably at 50° C. for 14 hours. One skilled in the art will understand that temperatures and time can be selected as suits the sample in question and the characteristics of the antibody being produced.

As used herein, ‘antibodies’ include functionally active fragments, derivatives or analogues and may be, but are not limited to, polyclonal, monoclonal, bi-, tri- or tetra-valent antibodies, humanized or chimeric antibodies, single chain antibodies, such as single chain Fv fragments, Fab fragments, Fab′ and Fab′₂ fragments, anti-idiotypic (anti-Id) antibodies, and epitope-binding fragments of any of the above. These antibodies and their fragments may be naturally occurring, humanized, chimeric or CDR grafted antibodies and standard molecular biology techniques may be used to modify, add or delete amino acids or domains as desired. Humanized antibodies are antibody molecules from non-human species having one or more complementarity determining regions (CDRs) from the non-human species and a framework region from a human immunoglobulin molecule (see, for example, U.S. Pat. No. 5,585,089). The antibody molecules purified using the methods of the invention can be of any class (e.g. IgG, IgE, IgM, IgD and IgA) or subclass of immunoglobulin molecule.

The methods for creating these antibody molecules are well known in the art (see for example, Shrader et al., WO 92/02551; Ward et al., 1989, Nature, 341:544; Orlandi et al., 1989, Proc. Natl. Acad. Sci. USA, 86:3833; Riechmann et al., 1988, Nature, 322:323; Bird et al, 1988, Science, 242:423; Queen et al., U.S. Pat. No. 5,585,089; Adair, WO91/09967; Mountain and Adair, 1992, Biotechnol. Genet. Eng. Rev, 10:1-142; Vermna et al., 1998, Journal of Immunological Methods, 216:165-181).

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp77-96, Alan R Liss, Inc., 1985).

Chimeric antibodies are those antibodies encoded by immunoglobulin genes that have been genetically engineered so that the light and heavy chain genes are composed of immunoglobulin gene segments belonging to different species. These chimeric antibodies are likely to be less antigenic. Bivalent antibodies may be made by methods known in the art (Milstein et al., 1983, Nature 305:537-539; WO 93/08829, Traunecker et al., 1991, EMBO J. 10:3655-3659). Bi-, tri- and tetra-valent antibodies may comprise multiple specificities or may be monospecific (see for example WO 92/22853).

Antibody sequences may also be generated using single lymphocyte antibody methods based on the molecular cloning and expression of immunoglobulin variable region cDNAs generated from single lymphocytes that were selected for the production of specific antibodies such as described by Babcook, J. et al., 1996, Proc. Natl. Acad. Sci. USA 93(15):7843-7848 and in WO 92/02551. The latter methods rely on the isolation of individual antibody producing cells which are then clonally expanded followed by screening for those clones which are producing an antibody which recognises its cognate antigen, and, if desired, the subsequent identification of the sequence of their variable heavy (V_{H}) and light (V_{L}) chain genes. Alternatively, the cells producing antibody that recognises its cognate antigen may be cultured together followed by screening.

Antibodies prepared using the methods of the invention are most preferably humanised antibodies which may be linked to toxins, drugs, cytotoxic compounds, or polymers or other compounds which prolong the half-life of the antibody when administered to a patient.

Methods for the expression of recombinant proteins are well known in the art. Suitable examples of host cells for the expression of antibodies generated, for example, as described above, include bacteria such as gram positive or gram negative bacteria, e.g. E. coli, or yeast cells, e.g. S. cerevisiae, or mammalian cells, e.g. CHO cells and myeloma or hybridoma cell lines, e.g. NSO cells. Most preferably, in the methods of the invention, a recombinant antibody is produced in bacteria, e.g. E. coli (see Verma et al., 1988, J. Immunol. Methods 216:165-181; Simmons et al., 2002, J. Immunol. Methods 263:133-147).

E. coli host cells may be naturally occurring E. coli strains or mutated strains capable of producing recombinant proteins. Examples of specific host E. coli strains include MC4100, TG1, TG2, DHB4, DH5α, DH1, BL21, XL1Blue and JM109. Examples also include modified E. coli strains, for example metabolic mutants and protease deficient strains. One preferred E. coli host is E. coli W3110 (ATCC 27,325) a commonly used host strain for recombinant protein fermentations. The recombinant antibody produced using the methods of the present invention is typically expressed in either the periplasm of the E. coli host cell or in the host cell culture supernatant, depending on the nature of the protein and the scale of production. The methods for targeting proteins to these compartments are well known in the art, for a review see Makrides, Microbiological Reviews, 1996, 60, 512-538. Examples of suitable signal sequences to direct proteins to the periplasm of E. coli include the E. coli PhoA, OmpA, OmpT, LamB and OmpF signal sequences. Proteins may be targeted to the supernatant by relying on the natural secretory pathways or by the induction of limited leakage of the outer membrane to cause protein secretion examples of which are the use of the pelB leader, the protein A leader, the coexpression of bacteriocin release protein, the mitomycin-induced bacteriocin release protein along with the addition of glycine to the culture medium and the coexpression of the ki1 gene for membrane permeabilization. Most preferably, in the methods of the invention, the recombinant protein is expressed in the periplasm of the host E. coli.

Expression of the recombinant protein in the E. coli host cells may also be under the control of an inducible system, whereby the expression of the recombinant antibody in E. coli is under the control of an inducible promoter. Many inducible promoters suitable for use in E. coli are well known in the art and depending on the promoter, expression of the recombinant protein can be induced by varying factors such as temperature or the concentration of a particular substance in the growth medium (Baneyx, Current Opinion in Biotechnology, 1999, 10:411-421; Goldstein and Doi, 1995, Biotechnol. Annu. Rev, 105-128). Examples of inducible promoters include the E. coli lac, tac, and trc promoters which are inducible with lactose or the non-hydrolyzable lactose analog, isopropyl-β-D-1-thiogalactopyranoside (IPTG) and the phoA, trp and araBAD promoters which are induced by phosphate, tryptophan and L-arabinose respectively. Expression may be induced by, for example, the addition of an inducer or a change in temperature where induction is temperature dependent. Where induction of recombinant protein expression is achieved by the addition of an inducer to the culture the inducer may be added by any suitable method depending on the fermentation system and the inducer, for example, by single or multiple shot additions or by a gradual addition of inducer through a feed. It will be appreciated that there may be a delay between the addition of the inducer and the actual induction of protein expression for example where the inducer is lactose there may be a delay before induction of protein expression occurs while any pre-existing carbon source is utilized before lactose.

E. coli host cell cultures (fermentations) may be cultured in any medium that will support the growth of E. coli and expression of the recombinant protein. The medium may be any chemically defined medium, such as those provided in Pirt S. J. (1975) Principles of Microbe and Cell Cultivation, Blackwell Scientific Publications, with modifications where appropriate to control growth rate as described herein. An example of a suitable medium is ‘SM6E’ as described by Humphreys et al., 2002, Protein Expression and Purification, 26:309-320.

Culturing of the E. coli host cells can take place in any suitable container such as a shake flask or a fermenter depending on the scale of production required. Various large scale fermenters are available with a capacity of greater than 1,000 litres up to about 100,000 litres. Preferably fermenters of 1,000 to 50,000 litres are used, more preferably 1,000 to 10,000 litres. Smaller scale fermenters may also be used with a capacity of between 0.5 and 1,000 litres.

Fermentation of E. coli may be performed in any suitable system, for example continuous, batch or fed-batch mode (Thiry & Cingolani, 2002, Trends in Biotechnology, 20:103-105) depending on the protein and the yields required. Batch mode may be used with shot additions of nutrients or inducers where required. Alternatively, a fed-batch culture may be used and the cultures grown in batch mode pre-induction at the maximum specific growth rate that can be sustained using the nutrients initially present in the fermenter and one or more nutrient feed regimes used to control the growth rate until fermentation is complete. Fed-batch mode may also be used pre-induction to control the metabolism of the E. coli host cells and to allow higher cell densities to be reached (Lee, 1996, Tibtech, 14:98-105).

Preferred features of each embodiment of the invention are as for each of the other embodiments mutatis mutandis. All publications, including but not limited to patents and patent applications cited in this specification are herein incorporated by reference as if each individual publication were specifically and individually indicated to be incorporated by reference herein as though fully set forth.

The invention will now be described with reference to the following examples, which are merely illustrative and should not in any way be construed as limiting the scope of the present invention.

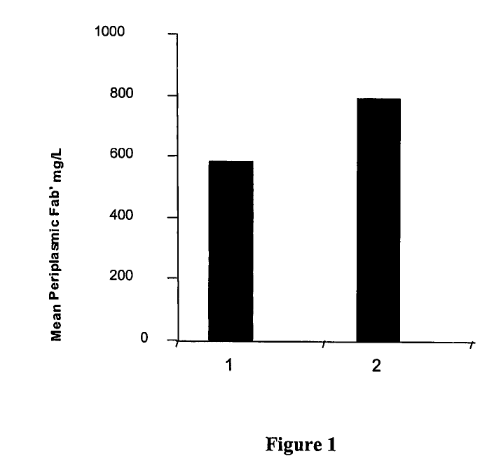

FIG. 1 is a histogram showing the effect of freeze-thaw treatment on the yield of functional antibody A. Numbers above each bar indicate functional antibody A yield in mg/litre clarified resuspension. Bar 1 shows the Fab′ yield from a control resuspension which was not frozen and bar 2 shows the Fab′ yield from a resuspension which was subjected to a freeze-thaw step.

### EXAMPLE 1

### Effect of Freeze-Thaw Treatment on Yield of Antibody A

Antibody A (a Fab′) was expressed in E. coli W3110 cells using the vector pTT0D with DNA encoding antibody A inserted. Fermentation (in DD53) was performed at 25° C. until OD₆₀₀ was 111.6 and ready for harvest. Fifty ml harvest culture aliquots at room temperature were centrifuged: one cell pellet was placed at −20° C. for 4 hours and a second pellet was resuspended in 5 ml of culture supernatant plus 29 ml H₂O and 5 ml of 1M Tris, pH 7.4 containing 100 MM EDTA before being subjected to heat treatment at 50° C. with agitation at 170 rpm for 14 hours. Post heat treatment, the resuspended cell pellets were clarified by centrifugation at 4200 rpm in a Beckman J.6 centrifuge for 30 mins at 4° C. Supernatant containing functional antibody A was assayed for Fab′ using Protein G HPLC analysis in 20 mM phosphate buffer. Antibody A was eluted using a pH gradient from pH 7.4 on injection, reducing to pH 2.5. Functional antibody yields were calculated by comparison with a standard Fab′ concentration.

An increase in yield of functional antibody can be seen in the frozen-thawed sample compared to no freeze-thawing (FIG. 1).

1. A method for the manufacture of recombinant antibody molecules comprising culturing a host cell sample transformed with an expression vector encoding a recombinant antibody molecule and subjecting said sample to a freeze-thaw treatment step.

2. The method according to claim 1, wherein the expression vector encodes at least part of an antibody light chain and at least part of an antibody heavy chain, such that at least some of the light and heavy chain antibody molecules are secreted and combine to form functional antibody.

3. The method according to claim 1 or claim 2, wherein the freeze-thaw treatment step consists of subjecting the sample to slow freezing and/or slow thawing.

4. The method according to any one of claims 1 to 3, wherein the frozen sample is stored between minus 20° C. and minus 70° C.

5. The method according to claim 4, wherein the frozen sample is subject to a temperature of minus 20° C.

6. The method according to claim 4, wherein the frozen sample is subject to a temperature of minus 70° C.

7. The method according to any one of the preceding claims, which additionally comprises at least one purification step, said purification step being performed after said method.

8. The method according to any one of the preceding claims, wherein the recombinant antibody molecule is a natural, humanised or chimeric antibody, or a fragment thereof.
